(19) United States
(12) Patent Application Publication
(10) Pub. No.: US 2019/0009062 A1
(43) Pub. Date: Jan. 10, 2019

(54) VESSEL WALL REPAIR SYSTEM

(71) Applicant: Lei LI, Beijing (CN)

(72) Inventor: Lei LI, Beijing (CN)

(73) Assignee: Evans Scientific (Beijing) Co., Ltd, Beijing (CN)

(21) Appl. No.: 16/064,791

(22) PCT Filed: Oct. 19, 2016
(86) PCT No.: PCT/CN2016/102619

§ 371 Date: Jun. 21, 2018

(30) Foreign Application Priority Data
Dec. 25, 2015 (CN) 201510998171.2

(51) Int. Cl. A61M 25/10; A61M 31/00; A61L 27/54; A61L 31/16

(52) U.S. Cl. CPC A61M 25/10; A61M 31/00; A61L 31/10; A61L 31/16; A61L 27/54

(57) ABSTRACT

Provided is a vessel wall repair system. The system comprises a repair member and a transporter that can detachably bear the repair member. A first bonding layer is disposed on a first side of the repair member facing to a vessel wall. After the transporter transports the repair member to a position to be repaired, the transporter can make the repair member press against the vessel wall to enable the repair member to be bonded to the vessel wall by means of the first bonding layer. In the system, a solution is innovatively used in which the repair member is directly bonded to a vessel wall in the repair of the vessel wall, and the system has a simple structure, is convenient to operate and has low cost.

### TECHNICAL FIELD

The present invention relates to the field of repair of vessel walls, and more particularly relates to a vessel wall repair system.

### BACKGROUND ART

Nowadays, cardiovascular and cerebrovascular diseases show multiple trends. Due to its own lesions and accidental injuries during interventional therapy, there will be damage to a vessel wall. For such damage, a covered stent-graft may be used for repair. The covered stent-graft technology involves the structural design of the stent-graft itself, as well as the design of an implantation system of the covered stent-graft. The structure is complex, the operation is cumbersome, and the cost is high.

### SUMMARY OF THE INVENTION

The present invention is directed to a vessel wall repair system. The vessel wall repair system has a simple structure and is convenient to operate.

To this end, the present invention provides a vessel wall repair system. The system includes a repair member and a transporter that can detachably bear the repair member. A first bonding layer is disposed on a first side of the repair member facing to a vessel wall. After the transporter transports the repair member to a position to be repaired, the transporter can make the repair member press against the vessel wall to enable the repair member to be bonded to the vessel wall by means of the first bonding layer.

Preferably, the transporter is a balloon.

Preferably, a second bonding layer is disposed on a second side of the repair member opposite to the first side, the repair member is bonded to the transporter through the second bonding layer, and the bonding force of the second bonding layer is smaller than the bonding force of the first bonding layer.

Preferably, the repair member is made of polyvinylidene fluoride.

Preferably, the first bonding layer and/or the second bonding layer include(s) a biological colloid material.

Preferably, the first bonding layer includes a photo-curable material.

Preferably, the vessel wall repair system further includes a light source, the transporter is a light-transmitting balloon, and the light source is disposed within the light-transmitting balloon.

Preferably, the first bonding layer is covered with a detachable release film.

Preferably, the vessel wall repair system further includes a control guide wire connected to the release film, so as to detach the release film from the first bonding layer through the control guide wire.

Through the foregoing technical scheme of the present invention, a solution is innovatively used in which the repair member is directly bonded to a vessel wall in the repair of the vessel wall, and the system has a simple structure, is convenient to operate and has low cost.

Additional features and advantages of the present invention will be set forth in part in the following detailed description of the invention.

### BRIEF DESCRIPTION OF THE DRAWINGS

Drawings are used to provide further understandings of the present invention, and constitute a part of the description. The drawings and the following detailed description of the invention serve to explain the present invention and do not constitute a limitation to the present invention. In drawings:

FIG. 1 is a structure schematic diagram of a vessel wall repair system provided by a preferred implementation of the present invention;

FIG. 2 is a sectional structure schematic diagram of a repair member provided by a preferred implementation of the present invention.

### DRAWING REFERENCE SIGNS

| | | | |
|---|---|---|---|
| 1 | Repair member | 2 | Transporter |
| 3 | First bonding layer | 4 | Second bonding layer |
| 5 | Release film | 6 | Control guide wire |

### DETAILED DESCRIPTION OF THE INVENTION

Specific implementations of the present invention will be described with reference to the drawings in detail below. It will be appreciated that the specific implementations described herein are only used to illustrate and explain the present invention and are not intended to limit the present invention.

In the present invention, a preposition such as “inside and outside” is used to refer to the inside and outside of the contour of a corresponding component, unless otherwise specified. “Far and near” are defined on the basis of a human intervention point when a vessel wall is repaired, that is, a direction far from the intervention point is far, and a direction close to the intervention point is near.

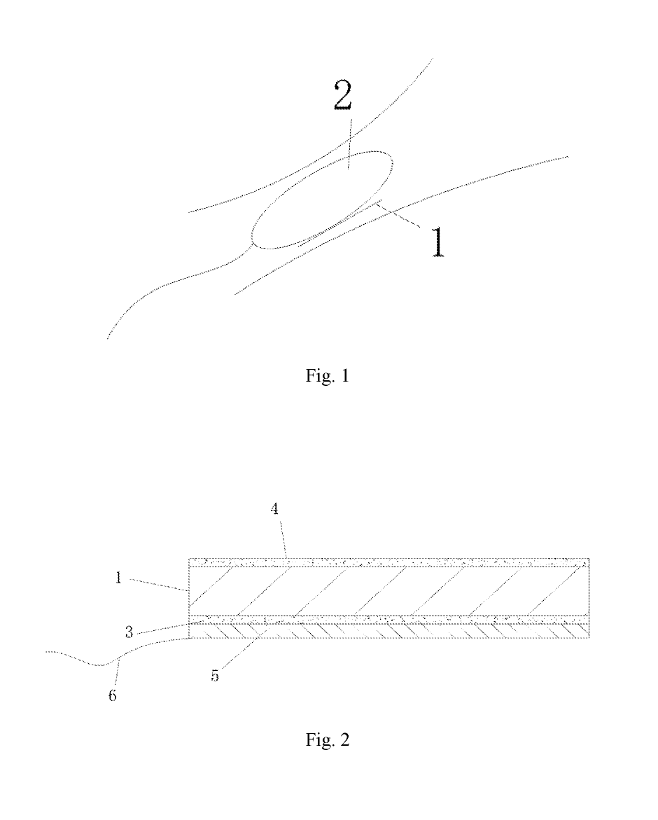

As shown in FIG. 1 and FIG. 2, an implementation of the present invention provides a vessel wall repair system. The system includes a repair member 1 and a transporter 2 that can detachably bear the repair member 1. A first bonding layer 3 is disposed on a first side of the repair member 1 facing to a vessel wall. After the transporter 2 transports the repair member 1 to a position to be repaired, the transporter 2 can make the repair member 1 press against the vessel wall to enable the repair member 1 to be bonded to the vessel wall by means of the first bonding layer 3. Therefore, in the present invention, a solution is innovatively used in which the repair member is directly bonded to a vessel wall in the repair of the vessel wall, and compared with a conventional covered stent-graft repair mode, the system has a simple structure, is convenient to operate and has low cost.

In the present implementation, in order to facilitate the bearing, transporting and pressing of the repair member 1, preferably, the transporter 2 is a balloon. When the balloon carries the repair member 1 to move to a vessel wall to be repaired, the operation of pressing the repair member 1 against the vessel wall may be completed by means of the expansion of the balloon. Thus, by means of the bonding property of the first bonding layer 3, the repair member 1 can be bonded to the vessel wall, so as to complete repair of the vessel wall. The balloon device is a well-known device in the field of interventional therapies, and the operations of moving, expanding, contracting and the like thereof in blood vessels are not described too much.

In addition, in order to detachably bear the repair member 1 on the transporter 2, preferably, as shown in FIG. 2, a second bonding layer 4 is disposed on a second side of the repair member 1 opposite to the first side, the repair member 1 is bonded to the transporter 2 through the second bonding layer 4, and the bonding force of the second bonding layer 4 is smaller than the bonding force of the first bonding layer 3. Therefore, when the repair member 1 is bonded to the vessel wall through the first bonding layer 3, the transporter 2 may be detached from the repair member 1.

In addition, in other implementations, there are many detachable bearing modes for the repair member 1 and the transporter 2, such as a sliding connection structure. Specifically, a receiving groove may be provided on the transporter 2. An opening that can allow the repair member 1 to slide out is provided on at least one end of the receiving groove in a movement direction of the transporter 2. When the repair member 1 is bonded to the vessel wall, it is only necessary to move the transporter 2 to a far or near side, so as to make the repair member 1 slide out of the receiving groove. The receiving groove may be a T-shaped groove or dovetail structure having a sliding connection function.

In the present implementation, the repair member 1 may be made of a membrana tectoria material of a covered stent-graft in the prior art, such as polyvinylidene fluoride, so that the repair effect is achieved and there is no harm to a human body. In addition, a material having a bonding property and included in the first bonding layer 3 and the second bonding layer 4 may be various colloids harmless to the human body such as a biological colloid.

In addition, in some therapies, it is required that the repair member 1 can overcome the rebound of a blood vessel. To address this problem, the repair member provided by the present implementation can have a flexible characteristic in a normal state and can be cured when needed. This material is preferably a photo-curable material in the present invention, that is, a photo-curable material such as UV resin may be included in the repair member 1 or the first bonding layer 3. In this way, the irradiation of, for example, an ultraviolet LED light source is used to cure the repair member 1 on the blood vessel wall as needed, thereby overcoming the problem of rebound of the blood vessel.

Correspondingly, in order to achieve the irradiation of the repair member 1, the vessel wall repair system further includes a light source, for example, an LED capable of generating ultraviolet light. In addition, for ease of operation, the transporter 2 may be designed as a light-transmitting balloon, and thus the light source may be disposed within the light-transmitting balloon, so as to achieve the transportation of the repair member as well as the selective curing of the repair member. The light source may be controlled in wired and wireless manners, wherein involved lines may be arranged in air and liquid supply pipelines of the balloon. The balloon in the implementation of the present invention may be a carbon dioxide inflatable balloon.

In addition, in order to prevent the first bonding layer 3 from being bonded to other materials before not bonded to the vessel wall, preferably, the first bonding layer 3 is covered with a detachable release film 5. In this way, the release film 5 is removed only when it is necessary to perform the bonding action, so as to fully ensure the bonding effect of the repair member 1 and the vessel wall. Specifically, the vessel wall repair system further includes a control guide wire 6 connected to the release film 5, so as to detach the release film 5 from the first bonding layer 3 through the control guide wire 6. Specifically, a distal end of the control guide wire 6 is connected to the release film, and a proximal end extends to a human body. Thus, the release film may be detached from the first bonding layer 3 by operating, for example, dragging the external end.

The preferred implementations of the present invention have been described in detail above with reference to the drawings. However, the present invention is not limited to specific details in the above implementations. Within the technical concept of the present invention, various simple variations of the technical schemes of the present invention can be performed. These simple variations all fall within the protection scope of the present invention.

In addition, it should be noted that the specific technical features described in the above specific implementations can be combined in any suitable manner without contradiction. To avoid unnecessary duplication, the present invention will not further describe various possible combination manners.

In addition, any combination of various different implementations of the present invention may also be performed as long as it does not violate the idea of the present invention, and it should also be regarded as the disclosure of the present invention.

What is claimed is:

1-13. (canceled)

14. A vessel wall repair system, comprising a repair member and a transporter that can detachably bear the repair member, a first bonding layer being disposed on a first side of the repair member facing to a vessel wall, wherein after the transporter transports the repair member to a position to be repaired, the transporter can make the repair member press against the vessel wall to enable the repair member to be bonded to the vessel wall by means of the first bonding layer.

15. The vessel wall repair system according to claim 14, wherein the transporter is a balloon.

16. The vessel wall repair system according to claim 14, wherein a second bonding layer is disposed on a second side of the repair member opposite to the first side, the repair member is bonded to the transporter through the second bonding layer, and the bonding force of the second bonding layer is smaller than the bonding force of the first bonding layer.

17. The vessel wall repair system according to claim 15, wherein a second bonding layer is disposed on a second side of the repair member opposite to the first side, the repair member is bonded to the transporter through the second bonding layer, and the bonding force of the second bonding layer is smaller than the bonding force of the first bonding layer.

18. The vessel wall repair system according to claim 14, wherein the repair member is made of polyvinylidene fluoride.

19. The vessel wall repair system according to claim 14, wherein the first bonding layer and/or the second bonding layer comprise(s) a biological colloid material.

20. The vessel wall repair system according to claim 14, wherein the repair member and/or the first bonding layer comprise(s) a photo-curable material.

21. The vessel wall repair system according to claim 20, wherein the repair member and/or the first bonding layer comprise(s) a photo-curable material.

22. The vessel wall repair system according to claim 21, further comprising a light source, the transporter being a light-transmitting balloon, and the light source being disposed within the light-transmitting balloon.

23. The vessel wall repair system according to claim 14, wherein the first bonding layer is covered with a detachable release film.

24. The vessel wall repair system according to claim 20, wherein the first bonding layer is covered with a detachable release film.

25. The vessel wall repair system according to claim 24, further comprising a control guide wire connected to the release film, so as to detach the release film from the first bonding layer through the control guide wire.

26. The vessel wall repair system according to claim 25, further comprising a control guide wire connected to the release film, so as to detach the release film from the first bonding layer through the control guide wire.
